(19) United States
(12) Patent Application Publication
(10) Pub. No.: US 2011/0059415 A1
(43) Pub. Date: Mar. 10, 2011

(54) Method and device for laser machining biological tissue

(75) Inventor: Anton Kasenbacher, Traunstein (DE)

(73) Assignee: Dr. Anton Kasenbacher and Lumera Laser GmbH, Traunstein (DE)

(21) Appl. No.: 12/805,968

(22) Filed: Aug. 26, 2010

Related U.S. Application Data
(63) Continuation of application No. PCT/EP2009/001250, filed on Feb. 20, 2009.

(30) Foreign Application Priority Data
Feb. 29, 2008 (DE) 10 2008 011 811.7

(51) Int. Cl. A61C 3/02; A61B 18/20; A61C 5/04

(52) U.S. Cl. 433/29; 606/2; 433/215

(57) ABSTRACT

A method of laser machining biological tissue including the provision of a pulsed processing laser beam and the processing of tissue by radiation using the pulsed processing laser beam, wherein the processing laser beam has a wavelength of the laser pulses ranging between 700 nm and 1400 nm, a time duration of the laser pulses ranging between 5 ps and 100 ps, and an energy density of the laser pulses on the surface of the tissue ranging between 1.5 J/cm² and 7.5 J/cm².

### CROSS-REFERENCE TO RELATED APPLICATIONS

This application is a continuation application under 35 U.S.C. §111(a), and is based upon and claims the benefit of priorities from U.S. International Application No. PCT/EP2009/001250 filed on Feb. 20, 2009, and German Patent Application No. 10 2008 011 811.7 filed on Feb. 29, 2008, the entire contents of which are incorporated herein by reference.

### BACKGROUND

The present invention relates to a method for machining or working biological tissue and a laser machining apparatus for machining biological tissue.

One example of the field of application of the present invention is dentistry in which a method and a corresponding laser machining device can be put to use instead of a mechanical drill for the ablation or abrasion of dentine, particularly dentine infected with caries. It is, however, understood that the invention can likewise find application in lasering other kinds of biological tissue and types thereof, such as, for example, hard tissue, soft tissue and tissue fluids.

In dentistry, particularly in caries therapy, the chief aim is to replace the conventional drill apparatus completely or partly by a laser since, unlike the drill such as a turbine always necessitating mechanical contact of the drill with the area being lasered, a laser machining device permits contactless lasering and ablation in thus achieving the operation with greater accuracy and, especially, painless. With current mechanical methods, pain may be triggered particularly in the dental pulp by the transmission of vibration or heat, a combination of both often being the case with a mechanical turbine due to the vibration and rotation also creating friction heat. All of these undesirable effects can be avoided by making use of a laser machining device.

Recent years have witnessed testing a series of laser systems for use in dentistry. But in many cases and particularly with the first such systems concerned, undesirable thermal or other collateral effects were observed, or the ablation efficiency was inadequate. This applied especially to laser systems operating on the basis of pulsed laser beam sources with pulse widths ranging from nano- to microseconds, in making use of, for example, excimer lasers with wavelengths in the ultraviolet range or Er: YSGG (λ=2.7 μm) or ER:YAG lasers (λ=2.94 μm) in the infrared wavelength range.

It was not until the introduction of short-pulse laser systems in the picosecond (ps) or femtosecond (fs) range and wavelengths in the visible or near infrared spectral range that a substantial advancement was achieved. First experimental studies indicated that these systems make it possible to achieve high quality dental ablation results with an efficiency at least equal to the performance of a mechanical turbine.

U.S. Pat. No. 5,720,894 describes a method and a device for material ablation by means of a pulsed laser beam source. The ablation parameters to be selected as regards wavelength, pulse width, energy and repetition rate of the laser pulses are indicated mainly just with reference to the task concerned in that each laser pulse is intended to interact with a thin surface portion of the material such that a plasma is formed in the focal position of the laser beam. The cited parameters of the laser beam are indicated relatively wide ranging in amounting up to 50 mJ or, relative to the surface area, up to 15 J/cm². But it is to be feared that with such a high pulse energy involving very short to ultrashort laser pulses, values are attained as to power or intensity in the pulse maximum, in which harmful collateral effects materialize due to non-linear processes such as multi-photon ionization, particularly when more than three photons are involved. It is particularly the case that such high peak pulses can result in molecules of water being ionized or DNA molecules can be ionized, causing harm to the genetic substance. The document fails to indicate how such collateral harm can be avoided, it, more particularly, lacking indication of how the parameters of the laser beam are to be set, as is likewise true of remaining prior art.

It is noted herewith that within the present application text the terms “laser machining”, “laser working” or lasering” are used with equal or synonymous meanings.

### SUMMARY

It is thus an object of the present invention to define a method and a device for laser machining biological tissue which now assures high efficiency whilst minimizing simultaneous collateral harm of the tissue area being lasered and its ambience.

This object is achieved by the features of the independent claims. Advantageous further embodiments and aspects read from the sub-claims.

One salient discovery of the present invention involves now making it possible to define a method and a device for laser machining biological tissue with adequate efficiency whilst ensuring that substantially no multi-photon ionization processes can take place involving more than three photons with the significance that the water molecules in the tissue are no longer ionized in lasering in thus avoiding effects deleterious to health. In addition, this also avoids ionization of DNA molecules in the cited multi-photon ionization processes involving more than three photons and thus harm to the genetic substance. The invention achieves this by defining a set of parameters of a pulsed laser beam.

In a first aspect of the present invention, a method for machining biological tissue is recited comprising providing a pulsed laser beam and machining tissue by beaming it with the pulsed laser beam configured so that laser pulses have a wavelength ranging from 700 to 1400 nm, a time duration ranging from 5 to 100 ps and an energy density on a surface of the issue ranging from 1.5 to 7.5 J/cm². The time duration can, for example, be measured in terms of the full width at half maximum (FWHM) of the laser pulse.

In one embodiment, the energy of the laser pulses can be set in a range below 100 μJ and the focal position of the laser beam on a tissue surface can be set with a diameter ranging from 10 to 100 μm.

It is understood that all parameter ranges stated in the present application also include all incremental intermediate values contained therein in disclosing the content of the present application.

In another embodiment, the method and the laser machining device can be put to use for ablating or abrading dentine, particularly when carious.

In yet another embodiment the pulse peak intensity of the laser pulses ranges from 10¹¹ to 1.5·10¹² W/cm².

In still a further embodiment it may furthermore be provided for that the repetition rate of the laser pulses ranges from 500 to 1000 Hz.

It is already known that in machining or laser machining biological tissue with a short-pulse laser such as a picosecond (ps) or femtosecond (fs) laser a microplasma is generated in a thin surface layer at the focal position of the laser beam which is ablated in a period of time of nanoseconds or microseconds in which the material is ejected from the beamed surface area by the transfer of energy of the quasi-free electrons generated by absorption or impact ionization at the atoms and molecules of the tissue. Experimental studies carried out on dentine have proven that, contrary to the results cited in prior art, laser machining is now possible with relatively moderate values as to energy density, energy or pulse peak power or intensity. Within the range of the parameters as cited above the microplasma is now generated slightly above the threshold at which the plasma or ablation is generated, resulting in the invention now making it possible to implement ablation with maximized compatibility both medically and biologically without running the risk of collateral harm. More particularly, the cited parameter ranges now avoid multi-photon ionization involving more than three photons in thus achieving that water molecules in the tissue are no longer ionized or at least to a safely negligible extent. However, any further increase of these values can risk collateral harm with no appreciable increase in ablation efficiency.

It is generally the case that a defined surface area of the biological tissue is worked with the machining laser beam and thus in a further embodiment the surface area is suitably scanned by the laser beam. Where this is concerned, it has been discovered to be an advantage when the machining laser beam has a top hat beam profile so that each partial area focussed by the laser beam is then scanned with precisely one laser pulse. However, it is just as possible to achieve this irrespective of whether a top hat profile is provided or not, by, for example, defining scanning such that respective adjoining partial areas are exposed to a single laser pulse with an overlap having a surface area smaller than half or smaller than some other fraction of the surface area of one partial area. In this way it can be achieved even when the “cross-section of the laser beam” has a Gauβian profile that a partial area hit substantially by the focus of the laser beam is exposed substantially to a single laser pulse.

In another embodiment it may be provided for that during machining the spatial position of the focus is held constant on the surface of the area. This can be achieved by various types of autofocusing means as detailed further on.

In yet another embodiment it may be provided for that in addition or parallel to machining with the machining laser beam a diagnosis is performed, the result of which is analyzed and used for controlling the machining process. For this purpose the presence of a signal generated in the tissue area or its ambience and, where necessary, the strength of the signal can be detected. This signal may be, for example, an optical signal. In particular, in the case of a microplasma being generated, the radiation generated by the plasma can be provided as such a signal. However, it is also possible that the

second or higher harmonic of an electromagnetic radiation directed at the surface area is used as the signal. The electromagnetic radiation may be that of a particularly pulsed diagnostic laser beam, the laser pulses of which feature an energy density which is smaller than that needed for ablating or machining the tissue. Indeed, the machining laser beam and the diagnostic laser beam may be generated by one and the same laser beam source switched back and forth between two operating modes.

In still another embodiment it may also be provided for that the signal is an acoustic signal as will be detailed further on.

The detection of the signal carried out during diagnosis may be used to turn the machining laser beam ON or OFF, especially when it is detected that no signal is present or when it does not have sufficient signal strength.

In a second aspect of the invention a laser machining apparatus for machining biological tissue is provided, comprising a laser beam source for providing a pulsed machining laser beam and a means for focussing the machining laser beam, the source laser beam being arranged to set the laser pulses to have a wavelength ranging from 700 to 1400 nm, a time duration ranging from 5 to 100 ps and an energy density ranging from 1.5 to 7.5 J/cm². The time duration can, for example, be measured in terms of the full width at half maximum (FWHM) of the laser pulse.

In one embodiment thereof the laser machining device may be arranged as a dental laser machining device for ablating or abrading dental material, particularly when carious.

In another embodiment thereof the laser machining device may further comprise a beam shaper for producing a substantially top hat beam profile.

In still a further embodiment thereof the laser machining device may further comprise a scan unit for scanning an area of the tissue with the laser machining beam. The scan unit can be arranged such and particularly can comprise a scanning velocity such that a partial area hit by the focus of the laser machining beam is exposed to precisely one laser pulse. The scan unit may furthermore be arranged so that respective adjoining partial areas which are each hit by a single laser pulse, will have a spatial overlap with each other the area of which is smaller than half of the partial area or smaller than some other fraction of the partial area.

In yet a further embodiment thereof the laser machining device may comprise an autofocus unit for maintaining constant the spatial position of the focus on the surface of the tissue.

It may furthermore be provided for that a numerical aperture (NA) can be selected as large as possible when applicable to the given possibilities as to how the lens and autofocus unit are arranged and engineered, for, the larger the numerical aperture (NA) the smaller the achievable focal position (or focal volume) on the surface of the tissue being laser machined and the smaller the energy density of the laser pulses can be selected.

In a further embodiment the laser machining device may comprise a detection unit for detecting the presence of a signal generated in the tissue or in the ambience thereof and, where necessary, the signal strength. A control unit may be connected to the detection unit for turning the laser beam source ON/OFF as a function of a signal provided by the detection unit.

The detection unit may comprise an optical sensor designed to, for example, sense radiation by a plasma being generated by the machining or to sense a second or higher harmonic of an electromagnetic radiation beamed onto the tissue.

The detection unit may also comprise an acoustic sensor when the signal to be detected is an acoustic signal.

The control unit may also be arranged to set the laser beam source to a laser machining mode for generating the pulsed machining laser beam or diagnosis mode for generating the specially pulsed diagnostic laser beam not suitable for laser machining the tissue and/or the laser pulses of which have an energy density smaller than the energy needed for laser machining the tissue.

In still a further embodiment the laser machining device may be arranged as a dental laser machining device comprising a fixing means for fixing a laser beam out-coupling unit configured as a handpiece by its distal end relative to the tooth being machined.

### BRIEF DESCRIPTION OF THE DRAWINGS

The invention will now be detailed by way of exemplary embodiments with reference to the drawings in which

FIG. 1 is a schematic illustration of one embodiment of a laser machining device;

FIG. 2 is a schematic illustration of a further embodiment of a laser machining device; and

FIGS. 3A and 3B are schematic illustrations of two embodiments of a fixing element connected by its distal end to a laser beam outcoupling unit.

### DESCRIPTION OF EMBODIMENTS

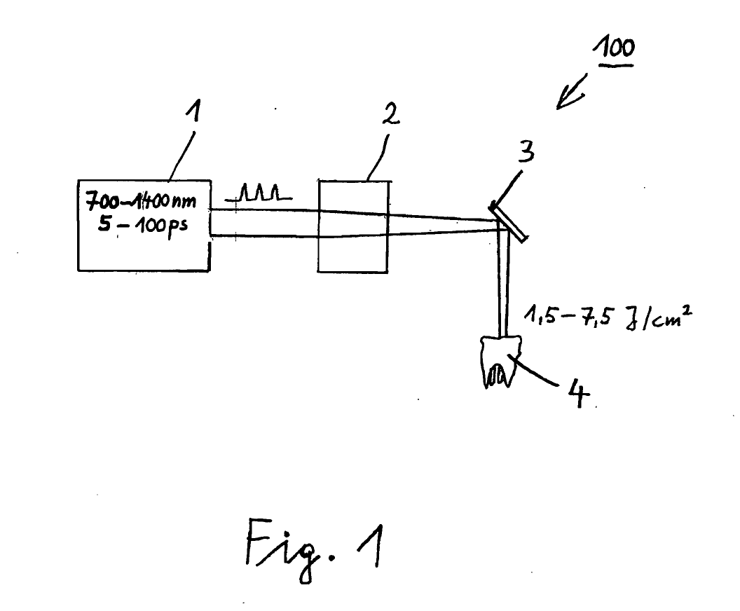

Referring now to FIG. 1 there is illustrated schematically a laser machining device 100 which as shown is a dental laser machining device for laser machining, abrading or ablating dental material, particularly when carious. It is understood, however, that the laser machining device may also be any other kind of medical laser machining device for laser machining some other kind of biological tissue.

The laser machining device 100 comprises a laser beam source 1 emitting a pulsed machining laser beam, the wavelength of which ranges from 700 to 1400 nm whilst the pulse duration of the laser pulses ranges from 5 to 100 ps.

The machining laser beam is directed at a focusing unit 2 by means of which the machining laser beam is focused on a patient's tooth 4 for laser machining. It may be necessary to first deflect the machining laser beam by an optical deflection unit 3 such as a mirror or a deflection prism. The machining laser beam is focussed so that the laser pulses have an energy density ranging from 1.5 to 7.5 J/cm² on the surface of the tooth 4.

It may be provided for that the laser beam source 1 generates the laser pulses such that the energy per pulse does not exceed 100 μJ. In this case the focusing unit 2 for maintaining the aforementioned energy density values is to be set so that the machining laser beam is focused at the surface of the tooth 4 with a diameter ranging from 10 to 100 μm.

It may furthermore be provided for that the laser beam source 1 emits the laser pulses with a repetition rate ranging from 500 Hz to 1000 kHz.

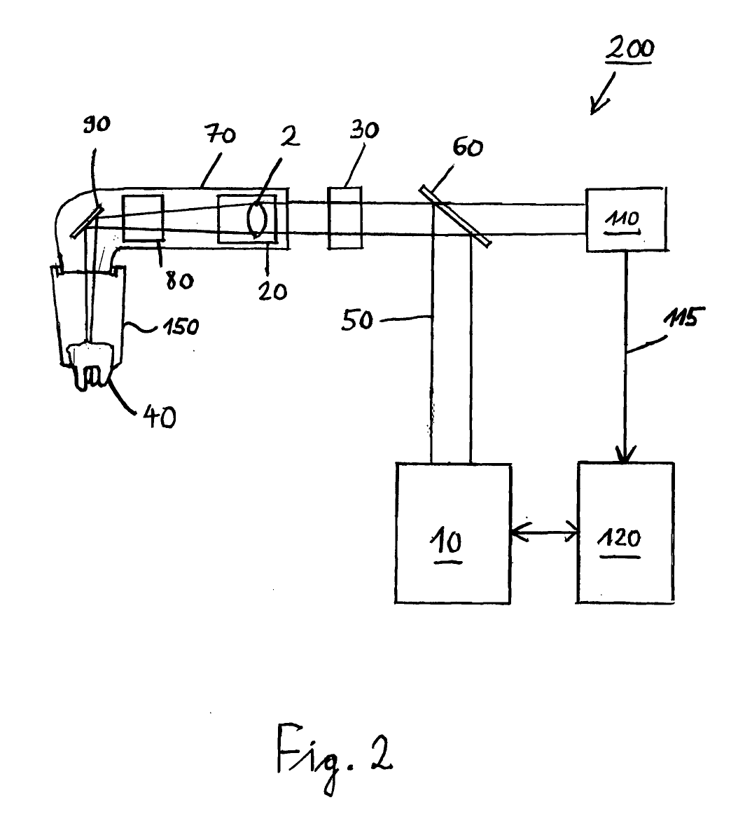

Referring now to FIG. 2 there is illustrated schematically a further embodiment of a laser machining device, not shown true to scale. The embodiment of a laser machining device 200 shown in FIG. 2 comprises a laser beam source 10 emitting a pulsed machining laser beam 50. In this embodiment as shown, the laser beam source 10 is a Nd:YAG laser coupled to a transient or regenerative amplifier emitting laser pulses in a wavelength of 1064 nm. It is understood that any other laser beam source such as, for example, a Nd:YVO4 or Nd:GdVO4 laser may be used. The pulse duration of the laser pulses is 10 ps and the repetition rate of the laser pulses ranges from 1 to 100 kHz. The energy of the laser pulses amounts to 40 μJ. At a repetition rate of 100 kHz the average radiation power is

### 4 W.

In the embodiment as shown in FIG. 2 the machining laser beam 50 emitted by the laser beam source 10 is directed at an optical deflection unit 60 which, however, only functions as a deflection unit for the wavelength of the machining laser beam 50, so that the machining laser beam 50 is deflected by approximately 90°.

In the light path of the machining laser beam 50 a subsequent beam shaping unit 30 is provided with which a top hat beam profile is generated.

Following this, the machining laser beam 50 enters am outcoupling unit 70 configured as a handpiece fronted by a lens 2 as part of an autofocusing unit 20 which by known means ensures that the focal position created by the lens 2 always remains within the plane of the lasered surface of the tooth 40. The autofocusing unit 20 may be combined particularly with a means for optically sensing from the radiation reflected by the surface of the tooth 40 whether the surface is still in the focal position of the machining laser beam. If it is not, a control signal is communicated to the autofocusing unit 20 to suitably result in the surface of the tooth 40 being returned into the focal position of the machining laser beam, for instance by moving the lens 2 forwards or backwards along the propagation path of the machining laser beam 50 by means of a fast stepper motor connected to a carriage mounting the lens 2. However, it is just as possible to configure the lens 2 so that its refraction is tweaked.

Referring still to FIG. 2 there is illustrated how the lens 2 is arranged so that it focuses the beam on the surface of the tooth 40 with a focal diameter of 40 μm. The laser pulse energy as recited above thus results in an energy density of 3.18 J/cm² which in turn produces a pulse peak intensity of 3.18·10¹¹ W/cm corresponding to a photon flux density of

1.7·10³⁰ photons·cm⁻²·s⁻¹. The electric field strength of the alternating electromagnetic field is 1.55·10⁷ V/cm and the average electron oscillation energy in the alternating electromagnetic field amounts to 0.021 eV.

It is understood that the beam shaper 30 may also be located in the beam path downstream of the lens 2, in other words particularly also in the outcoupling unit 70, although it is just as possible to combine the autofocusing unit 20 and beam shaper 30, especially the lens 2 and beam shaper 30 into a common optical component.

Arranged in a further portion of the outcoupling unit 70 is a scan unit 80 by means of which the machining laser beam 50, again as already known, for example, can be scanned by means of second rotating mirrors, each facing the other, over a defined area of the surface of the tooth 40 to be lasered. By means of a further deflection unit 50, such as, for example, a deflecting prism or a reflective mirror the machining laser beam 50 is then deflected in the direction of the tooth 40.

It is understood that although in this embodiment the scan unit 80 is arranged in the handpiece, other embodiments are just as possible in which the scan unit is located in the beam path upstream of the handpiece, i.e. particularly within an arm hinging the mirror or at the input thereto upstream of the handpiece.

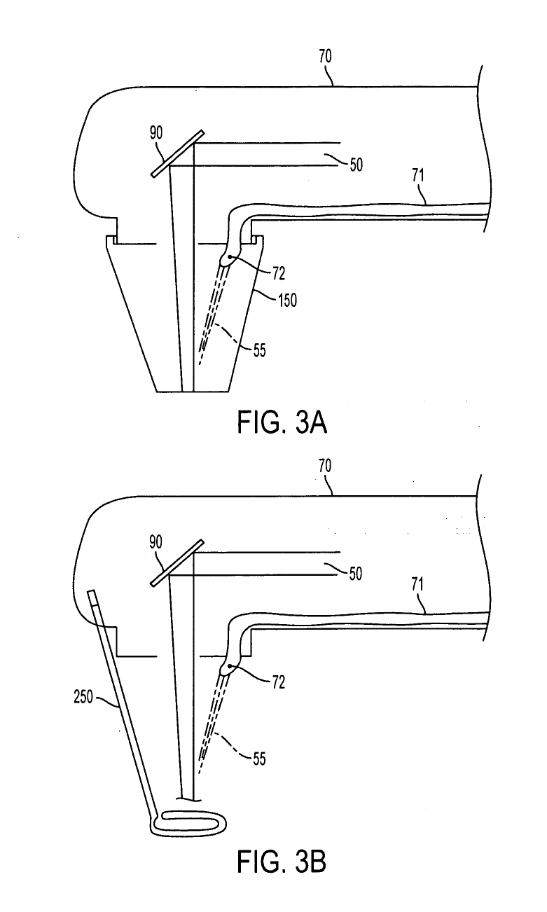

The outcoupling unit 70 configured as a handpiece needs to be held directed on the tooth being lasered by the physician. This is facilitated, particularly in maintaining the position of the distal end of the outcoupling unit 70 constant relative to the tooth 40 a funnel-shaped fixing element 150 is secured to the distal end of the outcoupling unit 70 and can be suitably located on the tooth 40 during lasering as detailed further on with reference to FIG. 3A.

Lasering may be additionally implemented in a predefined controlled atmosphere or under other predefined conditions such as air flow or the like.

The optical or acoustical signals generated in the lasered area of the surface of the tooth 40 or in the ambience thereof can be detected and used for diagnostic purposes. As regards optical signals it has already been explained that these are based, for example, either on the plasma radiation or second (SHG) or higher harmonic generated electromagnetic radiation acting on the dental material involved in lasering. The embodiment as shown in FIG. 2 will now be explained by way of the last-mentioned case of detecting a SHG signal.

In this mode of diagnosis a diagnostic laser beam is emitted, the energy or energy density of which is below the threshold for generating ablation or plasma so that no lasering occurs with the diagnostic laser beam which, like the machining laser beam is pulsed with the intention of diagnosing whether the partial area of the dental material to be lasered is carious or not. If not, it furnishes a higher SHG signal than carious dental material. The frequency doubled radiation generated as such at the surface of the tooth passes through the machining laser beam path in the opposite direction, at least in part, as described above, in other words is deflected by the deflection unit 90, passes through the scan unit 80, the autofocusing unit 20 with the lens 2 to finally incident the beam splitter 60 which, however, is transparent for the wavelength of the SHG signal so that the frequency doubled radiation can be input in an optical detection unit 110. The optical detection unit 110 may be a simple photo detector detecting the intensity of the SHG radiation. It is just as possible to use a more complex system such as a spectrometer, CCD camera or CMOS image sensor as the optical detector 110. Such sophisticated optical detectors may suitably serve to be used in combination with the autofocusing unit 20 as already indicated above.

The SHG radiation values detected by the optical detector 110 are converted into a signal 115 and input in a combined

analyzer/controller 120 which in this embodiment may also be a computer system. It is understood, however, that in principle any other type of control system is compatible, for instance, memory-programmable controllers, micro controllers or analog closed-loop controls.

The analyzer/controller 120 can be supplied with a signal from the laser beam source 10 containing data as to the operation status of the laser beam source 10. The analyzer/controller 120 outputs as a function of signal 115 communicated by the optical detector 110 a control signal which is input in the laser beam source 10 in switching it, for example, from an idle mode to a lasering mode, for instance, when the optical detector 110 communicates a corresponding signal 115 to the analyzer/controller 120 because of a low SHG signal indicating a carious condition.

The embodiment as shown in FIG. 2 comprises in particular a laser beam source 10 which is fast in mode switching between “OFF” (idle), “diagnosis” and “therapy” (lasering), where “fast” means—for example, in less than 0.1 second—that both the patient and the physician notice no interruption in treatment.

In this embodiment the laser beam source 10 emits both the machining laser beam during the “therapy” mode and the diagnostic laser beam during the “diagnosis” mode with the difference substantially in the energy density per pulse applied to the tooth in J/cm² which in the “diagnosis” mode needs to be reliably below the ablation threshold whilst in the “therapy” mode it is above this threshold.

A distinction can be made between the methods for fast mode switching within and outside of the laser. Switching is usually done by means of acousto-optical or electro-optical modulators. In acousto-optical modulation the machining laser beam is deflected by the refraction of a refractive index grating whereas in electro-optical modulation the polarization is turned in combination with a polarizer to produce the modulation. With optical amplifiers a distinction is made between transient single-pass or multiple-pass amplifiers whilst regenerative amplifiers feature an amplifier resonator.

The laser beam source used may include, for example, a transient amplifier and an electro-optical modulator (EOM) may be inserted between the picosecond (ps) oscillator and the amplifier. The circuitry involved achieves high voltage ON/OFF of the EOM (for example a Pockels cell) within 4-5 ns. In addition, the system is also capable of generating bursts of laser pulses, i.e. more than one amplified picosecond (ps) pulse per switching cycle.

Thus, the following variants are available for fast mode switching:

A) Selecting or Switching of the Repetition Rate

Selecting the repetition rate changes the pulse energy per pulse. As a result of how the laser functions the pulse energy of high repetition rates is less than that of lower repetition rates. The therapy mode then has a low repetition rate of, for example, 100 kHz whilst the diagnosis mode runs at a high repetition rate of for

example 500 kHz. In this arrangement the difference in the pulse energy between the two modes may amount to a certain factor possibly depending on the swing in the repetition rate and the laser beam source being used. This factor may range, for example from 1 to 10, for example 3 to 5 or some other factor. Making this selection is done in principle precise to a pulse, i.e. from one pulse to the next and how nimble it actually occurs depending on how software and hardware are signal processed, typically being 100 ns. The repetition rate can be selected faster than 1 ms or even faster than 1 μs.

B) Selecting or Switching to Burst Mode from Single-Pulse Operation

Here, with a fixed repetition rate of, for example, 100 kHz the energy of the laser pulse in single pulse operation is distributed over several pulses so that the energy of the pulses in the burst is below the ablation threshold, thus resulting in single-pulse operation being selected in the therapy mode whilst burst operation is selected in the diagnosis mode. The same as in case A) this selection is done in principle precise to a pulse and is likewise limited by the signal processing of the software and hardware. Accordingly, selection is likewise faster than 1 μs.

C) EOM High Voltage (HV) ON/OFF

This method permits with current best HV power supply components switching times better than 1 ms. The drawback with such fast switching may be a deviation from the ideal top hat pulse as would be reflected by the pulses being optically distorted with, in addition to a spike in the pulse energy of the first pulses by the stored pumped energy in the amplifier on power up as much as a magnitude depending on the design of the amplifier.

D) Motorized Turning the Polarization

Together with a polarization-selective element the energy of the laser pulses can be controlled, as is standard in lasers of the applicant Lumera in achieving with improved motors or nimble reciprocating vanes, selection times up to approx. 10 ms.

The picosecond (ps) laser can be operated in the constant mode in achieving modulation of the beam output with further external modulators (AOM, EOM) which are then freely triggerable. The speed when using an external EOM corresponds to that of case A) and B) whilst in case C) with external modulation there is no disadvantage of the energy spiking of the first pulses.

It is understood that selecting a therapy mode or lasering mode and a diagnostic mode with the laser beam source with a change in the parameters thereof is viewed as a separate invention in its own right, independent of the main aspect of the present application, namely the parameters of the pulsed machining laser beam. This aspect thus relates to a device for laser machining biological tissue comprising a laser beam source for providing a pulsed machining laser beam and a pulsed diagnostic laser beam, the laser pulses of the latter have an energy which is below that as needed for tissue lasering or ablation. The laser machining device can optionally feature one or more further features as described in this application in conjunction with the main aspect. This aspect also applies to a method for laser machining biological tissue comprising providing a pulsed machining laser beam and lasering the tissue by beaming it with the pulsed machining laser beam and diagnosing the tissue with a pulsed diagnostic laser beam, the pulses of which have a pulse energy below that as needed for lasering or ablation of the tissue. The method may optionally comprise one or more further features as described in the present application in conjunction with the main aspect. It is understood that the further independent aspect described in this paragraph may also relate to

lasering other materials, a diagnostic or testing lasering beam then being employed to test and “strobe” to a certain extent certain properties of the material involved before it actually being lasered as a function of the test results.

In a further embodiment (not shown) it may also be provided for that use is made of an acoustic signal for detection, it having been discovered namely that in generating a plasma during the lasering mode a characteristic acoustic signal is produced. The same as detecting plasma radiation by an optical sensor its noise can be detected by an acoustic sensor for input to the analyzer/controller 120 for further processing. Here again, it is understood that detecting the presence of an acoustic signal and, where necessary, its signal strength is viewed as a separate invention in its own right. This aspect thus relates to a device for laser machining biological tissue comprising a laser beam source for providing a pulsed machining laser beam, and a detector for detecting an acoustic signal and controlling the laser beam source as a function of the acoustic signal and where necessary its signal strength. The laser machining device can optionally feature one or more further features as described in this application in conjunction with the main aspect. Again, this aspect likewise relates to a method for laser machining biological tissue involving providing a pulsed machining laser beam and lasering the tissue by beaming it with the pulsed machining laser beam and detecting an acoustic signal in controlling the laser beam source as a function of the acoustic signal and where necessary its signal strength. The method may optionally comprise one or more further features as described in the present application in conjunction with the main aspect.

Referring now to FIGS. 3A and 3B there are illustrated exemplary aspects detailing in perspective a fixing element or locator. In the exemplary aspect as shown in FIG. 3A a fixing element or locator 150 is configured as a funnel, for example, as a mount for securing to a distal end of the outcoupling unit 70 configured as a handpiece, e.g. screw-threaded. Depending on the lasering situation a mount having a selected length or shape or a suitable diameter at its bottom end may be selected, it, as shown, having a tapered diameter downwards. It is, however, just as possible, that a downwards flared diameter may be used. This funnel shape of the fixing element or locator offers, in addition to location, also the possibility of implementing treatment at a site or area isolated from the remaining oval cavity. In addition, the ablated material can be suctioned off by a controlled action for removal through the handpiece (not shown). Furthermore, a further medium 55 such as cooling air may be introduced through a feeder conduit 71 guided in the handpiece and directed through a lower opening of the handpiece at the tooth being treated. For this purpose an adjustable nozzle 72 may be provided at the opening for jetting the medium 55 to the treatment area.

In a further embodiment, microscopic particles, such as nanoparticles or the like, can be directed at the treatment site. These nanoparticles may be definedly shaped to interact as wanted with the material already or still to be ablated in lasering to thus favorably influence lasering as a whole, particularly including ablation. For example, the nanoparticles may be put to use to differentiate healthy tissue from diseased tissue, although they may also be employed in conjunction with such methods as photodynamic therapy (PDT), photopolymerisation (for example covering open pulpa by a compoareaof synthetic collagen fibers) or photocoagulation by their interaction with the tissue in promoting the wanted effects. Another advantageous effect of nanoparticles could involve enhancing the ablation efficiency with no change in the laser intensity or conversely in minimizing laser intensity without detrimenting ablation efficiency (depending on the priority involved). Since every physician is governed by the principle of minimizing collateral harm (primum non nocere) nanoparticles can also be used to further reduce any free electrons still existing to further enhance the biosafety.

The microscopic particles can range in size from 1 nm to

1 μm, for example. They can be delivered by enriching a medium 55 such as cooling air through the feeder conduit 71 and jetted by means of the nozzle 72 to the treatment site.

Here again, it is understood that delivering microscopic particles such as nano particles in laser machining biological tissue is viewed as a separate invention in its own right independent of the main aspect of the present application, namely the parameters of the pulsed machining laser beam. This aspect thus relates to a method for laser machining biological tissue involving providing a pulsed machining laser beam and lasering the tissue by beaming it with the pulsed machining laser beam supported by the delivery of microscopic particles such as nano particles to the tissue area being lasered. The method can optionally feature one or more further features as described in this application.

In the exemplary aspect as shown in FIG. 3B the locator or fixing element is configured as a wire clip 250 suitably secured to a distal end of the outcoupling unit 70 configured as a handpiece by it being guided through a hole in the outer wall of the handpiece, for instance. It substantially consists of a single piece of wire comprising an elongated linear portion merging at its bottom end in a further portion formed into a ring. In this arrangement, the ring-shaped portion is not fully closed. Instead, one end thereof ends spaced away from the point at which the linear portion translates into the ring-shaped portion, the diameter of which can thus be varied within certain limits. During lasering the ring-shaped portion serves to clasp the tooth concerned in thus ensuring safeguarding proper positioning and orientation of the distal end portion of the handpiece relative to the tooth being lasered.

Again, it is understood that the general arrangement of a locator for safeguarding the positioning of a handpiece relative to the area being lasered is viewed as a separate invention in its own right, independent of the main aspect of the present application, namely the parameters of the pulsed machining laser beam. This aspect thus relates to a locator designed to be secured between a laser beam outcoupling unit and an area of the biological tissue being lasered. The locator can optionally feature one or more further features as described in this application. This aspect may also relate to a laser beam outcoupling unit containing one such locator, particularly engineered integral with the locator.

What is claimed is:

1. A method of laser machining biological tissue comprising:
providing a pulsed machining laser beam; and
processing the tissue by beaming the tissue with the pulsed machining laser beam, laser pulses of the pulsed laser beam having
a wavelength ranging from 700 to 1400 nm,
a time duration ranging from 5 to 100 ps, and
an energy density ranging from 1.5 to 7.5 J/cm².

2. The method as set forth in claim 1, wherein energy of the laser pulses is set in a range below 100 μJ and a focal position of the machining laser beam on a tissue area is set with a diameter ranging from 10 to 100 μm.

3. The method as set forth in claim 1, wherein the repetition rate of the laser pulses ranges from 500 to 1000 Hz.

4. The method as set forth in claim 1 employed for ablation or abrasion of dental material.

5. The method as set forth in claim 1, wherein the machining laser beam comprises substantially a top hat beam profile.

6. The method as set forth in claim 1, wherein a machined area is scanned by the machining laser beam.

7. The method as set forth in claim 6, wherein a partial area hit by a focus of the machining laser beam is exposed to precisely one laser pulse.

8. The method as set forth in claim 7, wherein respective adjoining partial areas hit by a single laser pulse have a spatial overlap having an area smaller than half of the partial area.

9. The method as set forth in claim 1, wherein control is implemented so that during laser machining the focal position remains on a surface of the machined area.

10. The method as set forth in claim 1, further comprising detecting the presence of a signal generated in the tissue area or its ambience and, where necessary, the strength of the signal.

11. The method as set forth in claim 10, wherein as a function of the detection result, the machining laser beam is switched ON/OFF.

12. The method as set forth in claim 10, wherein the signal is an optical signal.

13. The method as set forth in claim 10, wherein a plasma is generated in lasering the site, and the signal is provided by a radiation generated by the plasma.

14. The method as set forth in claim 10, wherein the signal is a second or a higher harmonic of an electromagnetic radiation irradiating the area.

15. The method as set forth in claim 14, wherein the electromagnetic radiation is that of the machining laser beam.

16. The method as set forth in claim 14, wherein the electromagnetic radiation is that of a diagnostic laser beam the energy density of which at the surface of the tissue is smaller than the energy density needed for laser machining the tissue.

17. The method as set forth in claim 16, wherein the machining laser beam and the diagnostic laser beam are generated by one and the same laser beam source.

18. The method as set forth in claim 10, wherein the signal is an acoustic signal.

19. The method as set forth in claim 1, wherein the tissue is additionally exposed to a gaseous medium.

20. The method as set forth in claim 19, wherein the medium contains microscopic particles.

21. A laser machining device for laser machining biological tissue comprising:
a source that provides a pulsed machining laser beam; and
means for focussing the machining laser beam, the laser pulses of the pulsed laser beam having
a wavelength ranging from 700 to 1400 nm,
a time duration ranging from 5 to 100 ps, and
an energy density ranging from 1.5 to 7.5 J/cm².

22. The laser machining device as set forth in claim 21, wherein energy of the laser pulses is set in a range below 100 μJ and
a focal position of the machining laser beam on a tissue area is set with a diameter ranging from 10 to 100 μm.

23. The laser machining device as set forth in claim 21, wherein a repetition rate of the laser pulses is set ranging from 500 to 1000 Hz.

24. The laser machining device as set forth in claim 21, wherein the device is a dental laser machining device for the ablation or abrasion of dental material.

25. The laser machining device as set forth in claim 21, further comprising:
a laser beam outcoupling unit, and
a locator connected to the laser beam outcoupling unit, to locate the laser beam outcoupling unit by a distal end thereof relative to the tooth being lasered.

26. The laser machining device as set forth in claim 21, further comprising:
a beam shaper to produce a substantially top hat beam profile of the pulsed machining laser beam.

27. The laser machining device as set forth in claim 19, further comprising:
a scan unit to scan a tissue area with the machining laser beam.

28. The laser machining device as set forth in claim 27, wherein the scan unit is arranged such that a partial area focussed by the machining laser beam is lasered by precisely one laser pulse.

29. The laser machining device as set forth in claim 27, wherein the scan unit is arranged so that each adjoining partial area is lasered with a single laser pulse with an overlap having a surface area smaller than half a partial area.

30. The laser machining device as set forth in claim 21, further comprising:
an autofocuser to maintain a focal position on the surface of the tissue constant.

31. The laser machining device as set forth in claim 21, further comprising:
a detector to detect a presence of a signal generated in the tissue or in an ambience and, where necessary, the signal strength thereof.

32. The laser machining device as set forth in claim 31, further comprising
a controller, connected to the detector and to the laser beam source, to turn the laser beam source ON/OFF as a function of a signal furnished by the detector.

33. The laser machining device as set forth in claim 31, wherein the detector comprises an optical sensor.

34. The laser machining device as set forth in claim 33, wherein the optical sensor is designed to sense radiation generated by the plasma being lasered.

35. The laser machining device as set forth in claim 33, wherein the optical sensor is designed to sense a second or higher harmonic of an electromagnetic radiation beamed into the tissue.

36. The laser machining device as set forth in claim 31, wherein the detector comprises an acoustic sensor.

37. The laser machining device as set forth in claim 32, wherein the controller is engineered to set the laser beam source to a lasering mode for generating the pulsed machining laser beam or diagnosis mode, whereby
in the lasering mode the pulsed machining laser beam is generated and in the diagnosis mode a diagnostic laser beam is generated having an energy density smaller than the energy density needed for lasering the tissue.
